(19) United States
(12) Patent Application Publication
(10) Pub. No.: US 2017/0164839 A1
(43) Pub. Date: Jun. 15, 2017

(54) ACOUSTIC PROBE AND SUBJECT INFORMATION ACQUISITION APPARATUS

(71) Applicant: CANON KABUSHIKI KAISHA, Tokyo (JP)

(72) Inventor: Atsushi Kandori, Ebina-shi (JP)

(21) Appl. No.: 15/377,610

(22) Filed: Dec. 13, 2016

(30) Foreign Application Priority Data
Dec. 15, 2015 (JP) 2015-244076

(51) Int. Cl. A61B 5/00; A61B 8/14; A61B 8/00; G01S 15/89

(52) U.S. Cl. CPC A61B 5/0095; G01S 15/8965; A61B 8/14; A61B 8/4494

(57) ABSTRACT

An acoustic probe includes at least two acoustic transducers, a support film in which the at least two acoustic transducers are disposed, and a recessed support member configured to support the support film. The support film is deformable so that the at least two acoustic transducers can be moved conforming to a shape of a subject.

### BACKGROUND

Field of the Disclosure

The present disclosure relates to a photoacoustic probe capable of receiving a photoacoustic wave by a photoacoustic effect, an acoustic probe, such as an ultrasonic probe capable of receiving an ultrasonic wave, and a subject information acquisition apparatus using these probes. Hereinafter, the term “acoustic wave” is used to include a sound wave, an ultrasonic wave, a photoacoustic wave, etc., but may be represented by an ultrasonic wave.

Description of the Related Art

Photoacoustic imaging (PAI) is proposed as one of optical imaging techniques. The photoacoustic imaging is a technique to receive an acoustic wave generated upon irradiation of light (also called a “photoacoustic wave”) and generate image data from acquired reception signals (see U.S. Patent Application Publication No. 2007/0287912). The photoacoustic wave is generated when pulsed light from a light source is applied to a subject, such as a living body, and tissues which have absorbed energy of light propagated through the subject expand.

In order to efficiently acquire information from a wide range of a subject, it is necessary to dispose ultrasonic (acoustic) transducers for receiving acoustic waves in a large area. Since target parts of subjects have various shapes and sizes, ultrasonic (acoustic) transducers for receiving acoustic waves disposed on a plane cannot always efficiently receive acoustic waves from the subjects. Accordingly, what is needed is an acoustic probe capable of efficiently receiving acoustic waves conforming to various target parts of subjects.

### SUMMARY

In an aspect of the present disclosure, an acoustic probe includes at least two acoustic transducers, a support film in which the at least two acoustic transducers are disposed, a recessed support member configured to support the support film, and a position detection unit configured to acquire positions information of each of the at least two acoustic transducers with respect to the support member. The support film is deformable so that the at least two acoustic transducers are moved conforming to a shape of a subject.

Further features of the present disclosure will become apparent from the following description of exemplary embodiments with reference to the attached drawings.

### BRIEF DESCRIPTION OF THE DRAWINGS

FIGS. 1A and 1B are schematic perspective views of an acoustic probe according to an embodiment of the subject disclosure, and FIG. 1C is a schematic cross-sectional view of the acoustic probe according to the embodiment depicted in FIGS. 1A and 1B.

FIG. 2 is a schematic perspective view of an acoustic probe according to another embodiment of the subject disclosure.

FIG. 3A is a schematic perspective view of an acoustic probe according to yet another embodiment of the subject disclosure, and FIG. 3B is a schematic cross-sectional view of the acoustic probe according to the embodiment provided in FIG. 3A.

FIG. 4A is a schematic perspective view of an acoustic probe according to an embodiment of the subject disclosure, and FIG. 4B is a schematic top view of the acoustic probe according to the embodiment illustrated in FIG. 4A.

FIG. 5A is a schematic perspective view of an acoustic probe according to an embodiment of the subject disclosure, while FIG. 5B is a schematic cross-sectional view of the acoustic probe according to the embodiment provided in FIG. 5A.

FIG. 6A is a schematic perspective view of an acoustic probe according to another embodiment of the subject disclosure, and FIG. 6B is a schematic cross-sectional view of the acoustic probe according to the embodiment illustrated in FIG. 6A.

FIG. 7A is a schematic cross-sectional view of an acoustic probe according to an embodiment of the subject disclosure, and FIG. 7B is a schematic cross-sectional view of the acoustic probe according to the embodiment provided in FIG. 7A.

FIG. 8A is a schematic cross-sectional view of an acoustic probe according to an embodiment of the subject disclosure, with FIG. 8B providing a schematic cross-sectional view of the acoustic probe of FIG. 8A.

FIG. 9 is a partial schematic cross-sectional view of an acoustic probe according to another embodiment of the subject disclosure.

FIGS. 10A and 10B are explanatory views of an acoustic probe according to additional embodiment of the subject disclosure, and FIG. 10C is a drive detection circuit diagram of the acoustic probe according to the embodiment detailed in FIGS. 10A and 10B.

FIG. 11 is a schematic diagram of a subject information acquisition apparatus according to an embodiment of the subject disclosure.

FIG. 12 is a schematic diagram of a subject information acquisition apparatus according to an embodiment of the subject disclosure.

FIG. 13 is a schematic diagram of a subject information acquisition apparatus according to an embodiment of the subject disclosure.

### DESCRIPTION OF THE EMBODIMENTS

A feature of an aspect of an acoustic probe of the present disclosure is that the acoustic probe includes a deformable support film with which acoustic transducers can be moved conforming to a shape of a subject. The support film may have any characteristics as long as, for example, it is deformable so that the acoustic transducers can be moved conforming to a shape of a subject upon reception of force and is restorable with its own restoring force or support force from a support member when the deforming force is eliminated. It is only necessary that the support film has appropriate characteristics among flexibility, bendability, stretchability, elasticity, etc. The acoustic probe of the present disclosure can constitute a subject information acquisition apparatus together with a signal processing unit which converts a signal detected by the acoustic probe into a signal representing information about the subject, etc. The acoustic probe can detect a photoacoustic wave from a subject generated by a photoacoustic effect, detect an ultrasonic wave from the subject, or transmit and receive an ultrasonic wave to and from the subject.

Hereinafter, embodiments of the present disclosure will be described with reference to the drawings. The present disclosure is not limited to these embodiments, and various modifications and changes may be made without departing from the spirit and scope of the disclosure.

### First Embodiment

In a photoacoustic probe of the present embodiment, in order to move plural acoustic transducers conforming to a surface shape of a subject, the plural acoustic transducers are provided on a film which is a deformable support film. The film is supported by a recessed support member. The support member is desirably formed by a member which is hard to some degree and is elastic in order to apply tension to the film when the deformable film is pressed against the subject. Since the recessed support member does not need to accommodate a matching medium etc., the support member may have a structure with only a framework (e.g., a basket-like structure). A wiring structure to each acoustic transducer can be, for example, a structure of a wire hanging down toward the support member from each acoustic transducer. In a support member of a framework structure, a wire or a wiring bundle can be drawn to the outside through a space of a through hole of the framework.

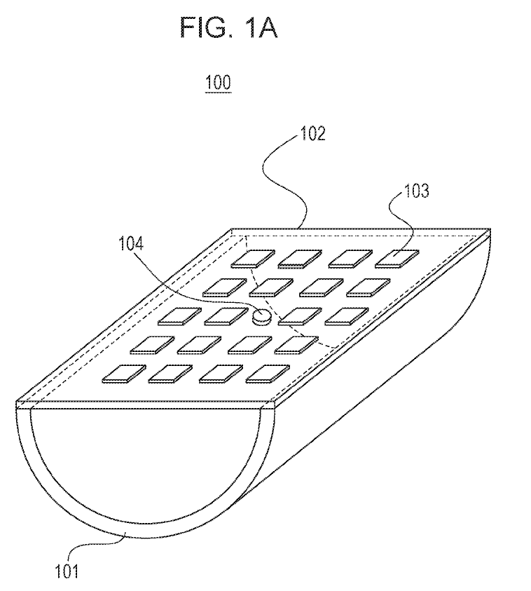

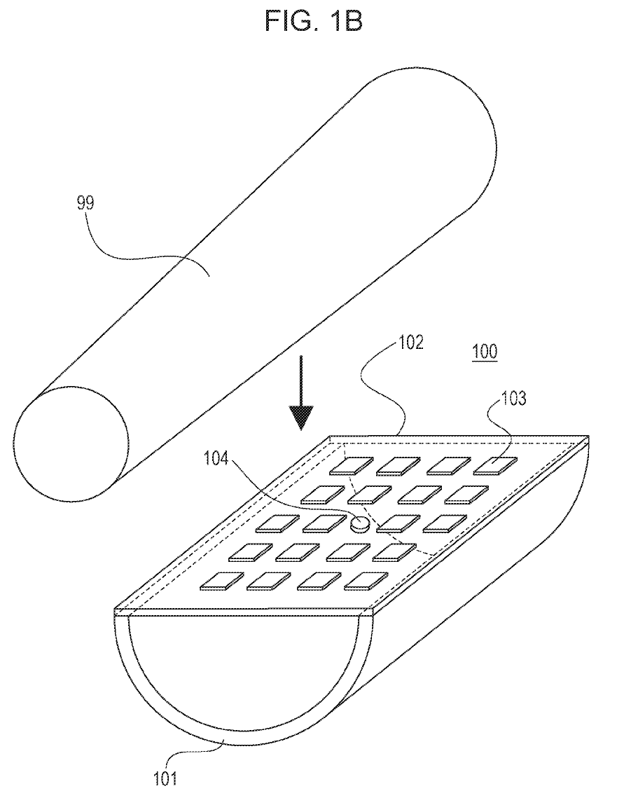

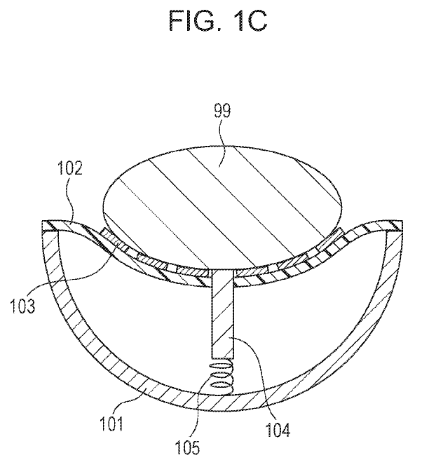

An acoustic probe 100 of the first embodiment will be described with reference to FIGS. 1A to 1C. In FIGS. 1A to 1C, the reference numeral 101 denotes a recessed support member, 102 denotes a flexible film, 103 denotes an ultrasonic (acoustic) transducer, and 104 denotes a light source as an illumination unit which illuminates a subject with light. A cable (not illustrated) as a bunch of electrical wires comes out from the support member 101. FIG. 1A is a schematic perspective view of an exemplary acoustic probe of the present embodiment. In FIG. 1A, the support member 101 has a recess (i.e., a groove) of a semilunar (semi-cylindrical) cross-section. The flexible film 102 is disposed to cover the recess of the support member 101, and an edge portion thereof is held by an end surface of the support member 101 which extends in parallel. In FIG. 1A, the flexible film 102 is held by the support member 101 from the right and left sides, and tension is applied to the film 102 itself (i.e., a tensioned state). A space is formed between the flexible film 102 as the support film and the recessed support member 101.

The ultrasonic (acoustic) transducers 103 for receiving acoustic waves are disposed on the flexible film 102 in a two dimensional array. The light source 104 is disposed in an area where no acoustic transducers 103 is disposed. The flexible film 102 is stretchable in areas where neither the acoustic transducers 103 nor the light source 104 is disposed. The acoustic transducers 103 supported by the flexible film 102 are movable in 5 axial directions (i.e., X, Y, Z directions (2 directions crossing in a film plane and a direction perpendicular to the film plane) and 2 directions inclined to a normal line of the acoustic transducer). Similarly, the light source 104 supported by the flexible film 102 is also movable in 5 axial directions.

When acquiring information from a subject 99, as illustrated in FIG. 1B, the subject 99 is pressed against the flexible film 102 of the acoustic probe (alternatively, the acoustic probe may be pressed against the subject 99). FIG. 1C is a cross-sectional view of the acoustic probe when acquiring information from the subject 99. Since the subject 99 is pressed against the flexible film 102 (i.e., against downward in FIG. 1C), the flexible film 102 is bent downward. The flexible film 102 in the areas where no acoustic transducers 103 are disposed is stretched instead of a spring, and absorbs a thickness of the subject 99. That is, these areas absorb a height and unevenness of a surface of the subject 99. Each acoustic transducer 103 moves downward in FIG. 1C corresponding to the flexible film 102, and the acoustic transducers 103 are automatically arranged conforming to a front shape of the subject 99. Orientations of the acoustic transducers 103 automatically change so as to substantially face the subject 99 with which the acoustic transducers 103 are in contact. In the acoustic probe of the present embodiment, by only pressing the subject 99 against the flexible film 102, the acoustic transducers 103 are automatically disposed conforming to the shape of the subject 99, and the orientations of the acoustic transducers 103 automatically change to face the subject 99.

If the acoustic transducers 103 are fixed to a certain surface, distances from the subject 99 to the acoustic transducers 103 may become nonuniform depending on the shape of the subject 99. If the distances from the subject 99 to the acoustic transducers 103 vary, an attenuation of an acoustic wave (an ultrasonic wave) changes in these portions and therefore, signals of the acoustic waves to receive decrease. In the acoustic probe of the present embodiment, since a magnitude of the distance between the subject 99 and each of the acoustic transducers 103 and uniformity in the distance can be reduced as small as possible, uniform acoustic waves with little attenuation can be received by the plural acoustic transducers 103.

If the distance between the subject 99 and each of the acoustic transducer 103 is large, the reception signal may deteriorate in that distance due to multiple reflection of the acoustic wave (multiple reflection between the surface of the subject 99 and a surface of each acoustic transducer 103). In the acoustic probe of the present embodiment, since the distance between the subject 99 and each acoustic transducer 103 can be made minimum, the acoustic wave can be received with a minimum influence of the multiple reflection. In addition, since the acoustic transducer 103 has directivity in receiving sensitivity characteristics, receiving sensitivity decreases when receiving an acoustic wave (an ultrasonic wave) from a direction of which angle is deviated from a direction to face the subject 99. Therefore, if the acoustic transducers 103 are fixed to a certain surface, sensitivities of the plural acoustic transducers 103 with respect to the subject 99 may become nonuniform depending on a shape of the subject 99. In the acoustic probe of the present embodiment, since the acoustic transducers 103 can face the subject 99, the acoustic wave can be acquired in a direction with sufficient receiving sensitivity efficiency of the acoustic transducers 103 with respect to the subject 99.

The support member 101 can be easily constituted by resin or metal. Although a thickness of the support member 101 is uniform in FIGS. 1A to 1C, the thickness of the support member 101 is not limited to the same. The support member may be formed in various shapes, such as a rectangular parallelepiped shape with a semilunar groove (recess). Although the recess of the support member 101 is a semilunar groove in the present embodiment, the shape of the recess is not limited to the same. The shape of the recess can be made to conform to the shape of the subject. If a semilunar groove is used, the probe is suitable especially to acquire information from an elongated subject, such as a finger, an arm, a leg, a neck, etc.

The flexible film 102 can be easily constituted by a resin film, rubber, etc., of a thickness enough to support the acoustic transducers 103. Specifically, epoxy resin, vinylchloride resin, polyester resin, fluorine-based resin, silicone rubber, etc. can be used. The flexible film 102 of from about 0.01 GPa to about 5 GPa in Young's modulus, and more preferably, from about 0.01 GPa to about 0.1 GPa in Young's modulus can be used. Other than these, materials deformable (stretchable, bendable, etc.) into substantially the same size of a subject when pressed by the subject can be used. Although a film of an uniform thickness is used in FIGS. 1A to 1C, the thickness of the film is not limited to the same. The film may have different thickness partially (e.g., the film is thinner in areas where no acoustic transducers 103 are disposed than in areas where the acoustic transducers 103 are disposed). Therefore, mobility of the acoustic transducers 103, bendability of the flexible film 102 by the subject 99, etc. can be adjusted optimally. For the ease of description, the film is referred to as the “flexible film 102” in most places in this specification. However, the film 102 is not limited to a flexible (or slightly elastic) film, and any other materials, such as an elastic body, may also be used. Being “flexible” is “soft and not to be folded discontinuously when being bent” and is similar to “elastic,” but “flexible” is not as highly stretchable as “elastic” and, in this sense, an expression “slight elastic” may also be used. Other than an elastic body, various materials with characteristics necessary for deformation, such as bending and stretching, can be used.

Any types of acoustic transducers 103 which can receive an acoustic wave (an ultrasonic wave) generated by illuminating the subject 99 with light can be used. Specifically, PZT, PVDF, CMUT, etc. which are acoustic transducers 103 generally used for an ultrasonic diagnostic apparatus can be used. In FIGS. 1A to 1C, the acoustic transducers 103 are disposed on a surface of the flexible film 102 on the side of the subject 99, but the configuration is not restrictive. The acoustic transducers 103 may be buried partially in or may penetrate the flexible film 102. If the flexible film 102 has the acoustic wave (ultrasonic wave) transmission characteristic with no problem of use, the acoustic transducers 103 may be disposed on a surface of the flexible film 102 on the reverse side of the subject 99 (i.e., a back side).

Any types of the light source 104 which can illuminate the subject 99 with light to generate an acoustic wave can be used. Although the light source 104 penetrates the flexible film 102 in FIGS. 1A to 1C, the configuration is not restrictive. The light source 104 may be disposed on the surface of the flexible film 102 or may be partially buried in the flexible film 102.

In the configuration of the example illustrated in FIG. 1C of the present embodiment, a spring 105 is provided between a bottom surface of the support member 101 and the light source 104, this configuration is not restrictive. By reducing the size and weight of the light source 104 using semiconductor laser, LED, etc. so that the light source 104 is supported by the flexible film 102, the spring 105 becomes unnecessary. Alternatively, light may be guided with an optical fiber, etc. from an external light source, and a light emitting end portion may be attached to the film 102 etc.

According to the present embodiment, a photoacoustic probe capable of efficiently receiving acoustic waves conforming to various target parts of subjects can be provided.

### Second Embodiment

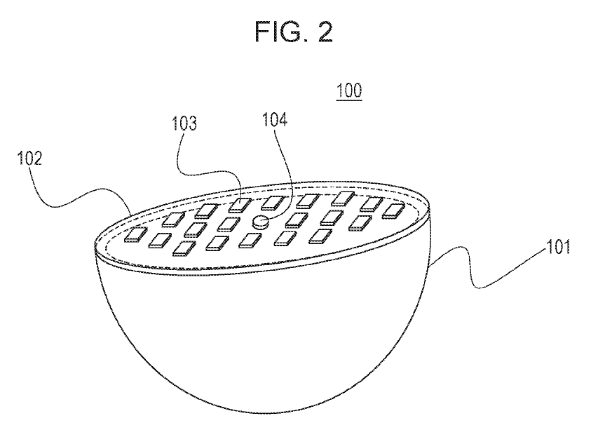

A second embodiment relates to a shape of a recessed support member. Other configurations are the same as those of the first embodiment. An acoustic probe related to the second embodiment will be described with reference to FIG. 2. A feature of the present embodiment is that a support member 101 has a hemispherical recess. In the present embodiment, since the support member 101 has a hemispherical recess, a subject can be surrounded by a flexible film 102 from more directions than in the first embodiment. Therefore, subject information can be acquired by pressing a protruding portion of the subject against the film 102. The present embodiment is suitable especially for acquiring subject information of a hand, a foot, an elbow, a knee, a breast, etc.

Although the shape of the recess of the support member 101 is hemispherical in the present embodiment, the shape of the support member 101 is not limited to the same. The support member 101 may also have a rectangular parallelepiped recess, a multi-sided pyramid shaped recess, a multi-sided pyramid stand shaped recess, an elliptical hemisphere shaped recess, etc.

### Third Embodiment

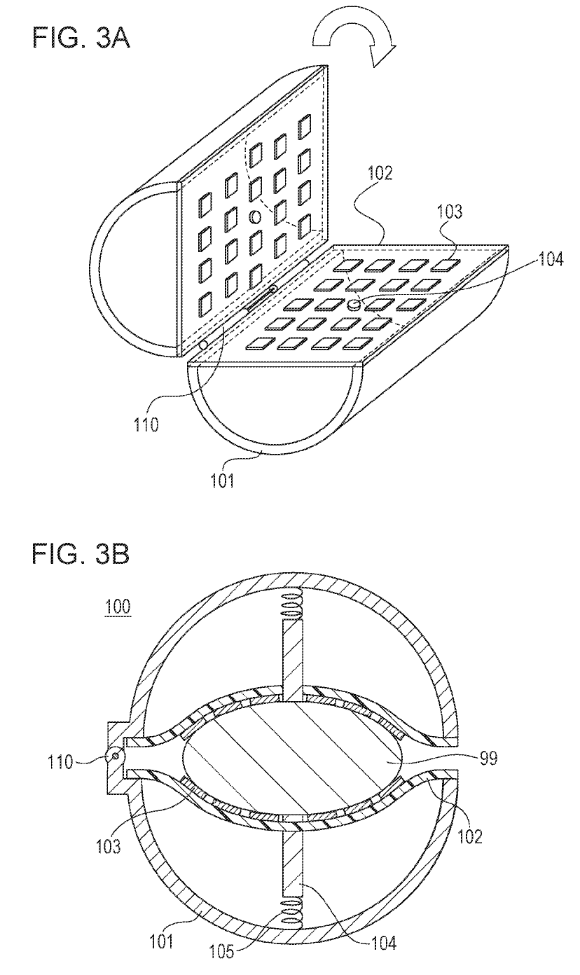

A third embodiment relates to the number of flexible films. Other configurations are the same as those of the first or the second embodiment. The third embodiment will be described with reference to FIGS. 3A and 3B. In FIGS. 3A and 3B, the reference numeral 110 denotes a hinge portion. A feature of the present embodiment is that plural (here, two) flexible films are provided to nip a subject from different directions. In particular, plural acoustic probes described in the first embodiment are provided and are joined at a hinge portion 110. In an exemplary configuration illustrated in FIG. 3A, two pairs of support films in which plural acoustic transducers are disposed and recessed support members which support the films are provided, and these two pairs are mutually movable (pivotable) so as to nip a subject between the support films. Photoacoustic waves from different surfaces, not only from one surface, of a subject 99 can be acquired simultaneously since the subject 99 is nipped between the films 102 of the plural acoustic probes and the plural probes are fixed mutually. Substantially all the photoacoustic waves can be acquired from the directions of about 360 degrees of an outer periphery of the subject 99. In the present embodiment, since the subject 99 is nipped by the acoustic probes, the subject 99 does not easily move during acquisition of information about the subject 99. Therefore, an acoustic wave including even more correct subject information can be received.

Although description is given with reference to the acoustic probe of the first embodiment, the present embodiment is not limited to the same. As long as the subject is nipped from different directions, plural acoustic probe of various forms including the acoustic probes described in the second embodiment can be used similarly.

### Fourth Embodiment

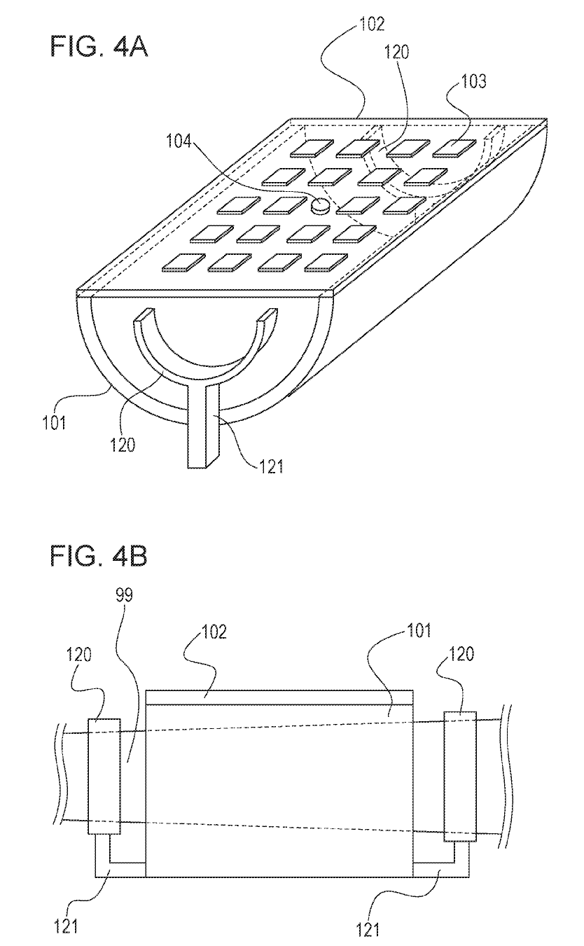

A fourth embodiment relates to a probe provided with a means for supporting a subject 99 (a retaining structure). Other configurations are the same as those of any of the first to the third embodiments. An acoustic probe of the present embodiment will be described with reference to FIGS. 4A and 4B. FIG. 4A is a schematic diagram of the acoustic probe of the present embodiment. FIG. 4B is a schematic diagram of the acoustic probe of the present embodiment seen from above. In FIGS. 4A and 4B, the reference numeral 120 denotes a pedestal, and 121 denotes a fixing portion which fixes the pedestals 120 to a support member 101.

A feature of the fourth embodiment is that the pedestals 120 for supporting a subject 99 are provided. In the present embodiment, the subject 99 is assumed to be elongated and the pedestals 120 are disposed at both ends of the support member 101. A positional relationship between the pedestal 120 having a U-shaped holder portion and the support member 101 is fixed via a fixing portion 121.

Since the present embodiment has the pedestals 120, when the subject 99 is pressed against a flexible film 102, the subject 99 can be supported by the pedestals 120. Therefore, a position of the subject 99 can be easily maintained stably. Since it becomes easier for the subject 99 to stay at the same position during acquisition of information from the subject 99, load applied to the subject 99 can be reduced. Since motion of the subject 99 can be reduced to the minimum, a shift of a positional relationship between the subject 99 and each of the plural acoustic transducers 103 during acquisition of the subject information can be reduced. Therefore, deterioration of a signal acquired from the subject 99 caused by a change in the positional relationship between the subject 99 and each of the plural acoustic transducers 103 can be reduced.

In the configuration of the present embodiment, it is not necessary that the flexible film 102 has a retaining function of the subject 99, and hardness of the flexible film 102 can be selected from a wider range. Specifically, hardness of the flexible film 102 can be lowered and, a film which conforms more closely to a surface shape of the subject 99 can be selected. Therefore, it is possible to automatically dispose the acoustic transducers 103 conforming more closely to the surface shape of the subject 99.

According to the acoustic probe of the present embodiment, during acquisition of information about the subject, the positional relationship between the subject and each of the acoustic transducers can be fixed easily. Therefore, a photoacoustic probe capable of efficiently receiving high quality acoustic waves conforming to various target parts of subjects can be provided.

### Fifth Embodiment

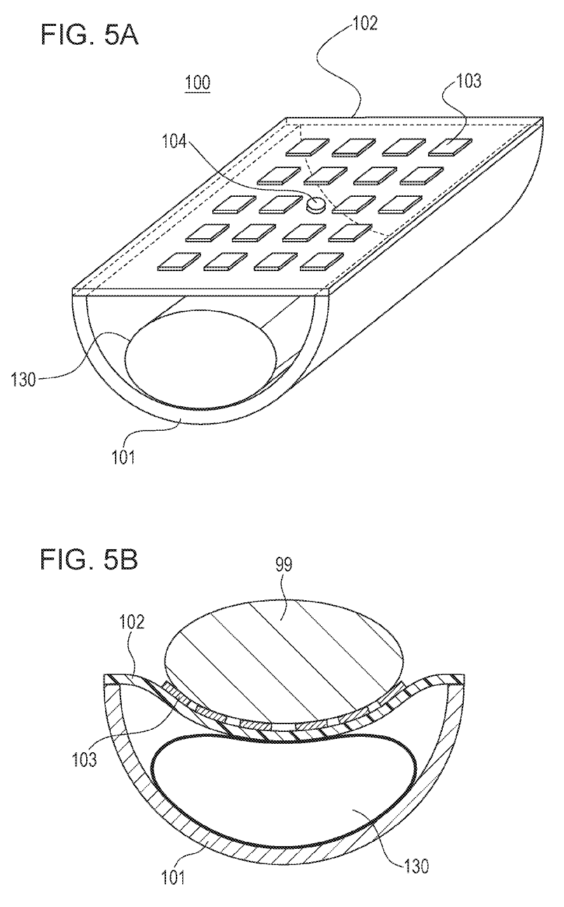

A fifth embodiment relates to a probe which has a means of another form for supporting a subject 99 (a retaining structure). Other configurations are the same as those of any of the first to the fourth embodiments. An acoustic probe of the present embodiment will be described with reference to FIGS. 5A and 5B. FIG. 5A is a schematic perspective view of the acoustic probe of the present embodiment. FIG. 5B is a schematic cross-sectional view of the acoustic probe of the present embodiment.

A feature of the fifth embodiment is that a bag 130 filled with gas for supporting a subject 99 is provided. The bag 130 filled with gas has a tubular shape, and is disposed at a bottom portion of a recess of the support member 101. The bag 130 is deformed when the subject 99 is pressed against the flexible film 102 as illustrated in FIG. 5B. Since the bag 130 is filled with gas, when the subject 99 is pushed to some degree (i.e., when the bag 130 is deformed to some degree), the bag 130 puts the subject 99 back. When the force putting the subject 99 back balances with the force pressing the subject 99 against the flexible film 102, the subject 99 is held at the position.

According to the present embodiment, since the bag 130 is used, the bag 130 can be deformed conforming to the surface shape of the subject 99, and the subject 99 is held in the surface. Therefore, load applied to the subject 99 can be reduced compared with a configuration which holds the subject 99 at several points by a hard member. Since motion of the subject 99 can be reduced to the minimum, a shift of a positional relationship between the subject 99 and each of the plural acoustic transducers 103 during acquisition of the subject information can be reduced. Therefore, deterioration of a signal acquired from the subject 99 caused by a change in the positional relationship between the subject 99 and each of the plural acoustic transducers 103 can be reduced. With the bag 130, an acoustic probe which can be flexibly used for subjects of various surface shapes, with which the need to select the shape of the subject is reduced, and which can maintain the position of the subject stably can be implemented.

According to the acoustic probe of the present embodiment, during acquisition of information about the subject, the positional relationship between the subject and each of the acoustic transducers can be fixed easily. Therefore, a photoacoustic probe capable of efficiently receiving high quality acoustic waves conforming to various target parts of subjects can be implemented.

### Sixth Embodiment

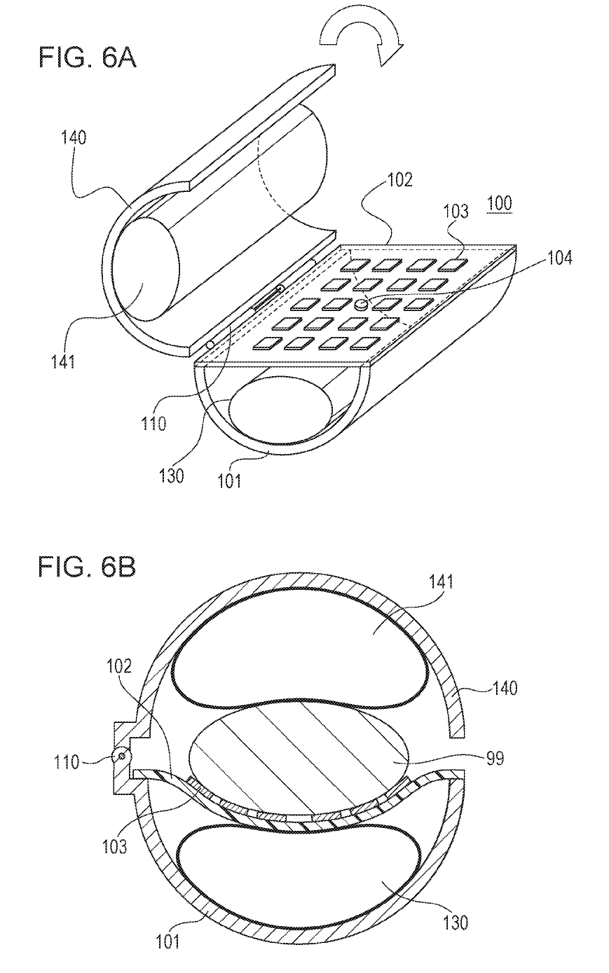

A sixth embodiment relates to a probe which has a means of an even another form for supporting a subject 99 (a retaining structure). Other configurations are the same as those of the fifth embodiment. An acoustic probe of the present embodiment will be described with reference to FIGS. 6A and 6B. FIG. 6A is a schematic diagram of the acoustic probe of the present embodiment. FIG. 6B is a schematic cross-sectional view of the acoustic probe of the present embodiment. In FIGS. 6A and 6B, the reference numeral 140 denotes a lid with a recess, and 141 denotes a stretchable bag.

A feature of the present embodiment is that the lid 140 of the same shape as a support member 101 of the acoustic probe is provided, and the lid 140 is attached to a part of the acoustic probe (a support member 101) via a hinge portion 110. A stretchable bag 141 in which gas is injected is provided in a recess of the lid 140. This exemplary configuration has a lid provided to be movable (pivotable) with respect to the support member 101, and the retaining means (the bag containing gas) are disposed inside the lid and in the space between the support film and the support member, respectively. The subject 99 is fixed by being nipped between the support member 101 and the lid 140 during acquisition of information about the subject 99. Then, gas is injected into the stretchable bag 141 and the bag 141 is expanded. Gas is injected until a space between the subject 99 and the lid 140 is filled with the stretchable bag 141, and the subject 99 is pressed against a flexible film 102 by the stretchable bag 141. An amount of gas injected into the stretchable bag 141 may be determined while monitoring internal pressure to be an optimal amount at which the subject 99 is fixed and is not moved and the subject 99 does not feel an unpleasant pain.

According to the acoustic probe of the present embodiment, during acquisition of information about the subject, the positional relationship between the subject and each of the acoustic transducers can be fixed easily. Therefore, high quality acoustic waves can be received efficiently conforming to various target parts of the subjects. Although the present embodiment is described with reference to the configuration of the fifth embodiment, the configuration is not restrictive. The present embodiment is applicable to various other configurations, including the configurations of the fourth embodiment and the first embodiment.

A stretchable bag 141 may be provided instead of the bag 130 filled with gas on the support member 101. In that case, in the same manner as in the stretchable bag 141 on the side of the lid, gas is injected into the bag on the side of the support member 101, and the subject 99 can be nipped from both vertical directions. In this configuration, since the subject 99 is held with certain force from both vertical directions, the subject 99 can be kept more stably.

### Seventh Embodiment

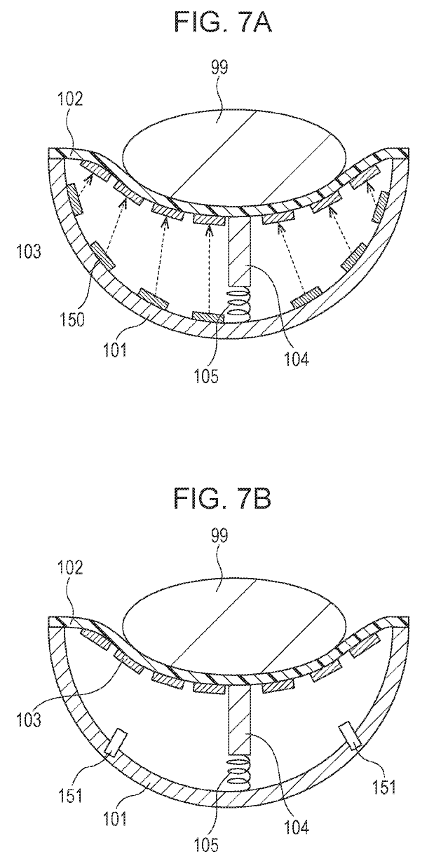

A seventh embodiment relates to a probe which has a position detecting function of acoustic transducers 103. Other configurations are the same as those of any of the first to the sixth embodiments. The present embodiment will be described with reference to FIGS. 7A and 7B. FIG. 7A is a schematic cross-sectional view of the acoustic probe of the present embodiment. In FIG. 7A, the reference numeral 150 denotes a position detection unit of the acoustic transducers 103.

A feature of the seventh embodiment is that the position detection units 150 which detects positions of the acoustic transducers 103 provided on a flexible film 102 with respect to a support member 101. When changing a signal received by the acoustic transducer 103 into subject information, it is necessary to reproduce the signal in consideration of the position of each acoustic transducer 103. Since coordinates of the acoustic transducers 103 of the present embodiment can be changed conforming to a surface shape of the subject 99, position coordinates of the plural acoustic transducers 103 change significantly depending on the subject 99. Therefore, estimated position coordinates of the acoustic transducers 103 can be considered by grasping the shape of the subject 99, etc. in advance during reproduction of information, but the reproduced subject information may become incorrect if the position coordinates of the acoustic transducers 103 shift greatly from the estimated positions.

In the present embodiment, it is possible to locate the coordinate position of each acoustic transducer 103 based on the support member 101. Therefore, when converting into (i.e., reproducing) subject information, correct position coordinate information about each acoustic transducer 103 can be used, whereby more correct information subject can be reproduced. According to the present embodiment, an acoustic probe capable of efficiently receiving acoustic waves conforming to various target parts of subjects, and correctly grasping coordinates of acoustic transducers during reception can be provided. More correct subject information can be reproduced by using the acoustic probe of the present embodiment.

A specific configuration of the position detection units 150 of the acoustic transducers 103 according to the present embodiment will be described with reference to FIG. 7B. In FIG. 7B, cameras 151 for acquiring images are provided as the position detection units 150 in a recess of the support member 101. The cameras 151 for acquiring images face the flexible film 102 and can acquire an image of each acoustic transducer 103. The cameras 151 of the probe can detect a position and a condition of each acoustic transducer 103 based on acquired image data, and can calculate position coordinates and inclination.

An image identification pattern for identifying its own self may be disposed on a back side (i.e., a surface on the side opposite to the side on which the subject 99 exists) of the acoustic transducer 103. Therefore, a position and an angle of the acoustic transducer 103 can be grasped more correctly. Every acoustic transducer 103 may have a different image identification pattern. In that case, since individual acoustic transducer 103 can be discriminated easily, it becomes less likely that each acoustic transducer 103 is erroneously recognized, and the position of the acoustic transducer, etc. can be grasped more correctly.

According to the configuration of FIG. 7B, a unit with which the position of the acoustic transducer 103 can be detected is implemented with a simple configuration. Therefore, a small-sized or lightweight photoacoustic probe capable of efficiently receiving acoustic waves conforming to various target parts of subjects, and correctly grasping coordinates of acoustic transducers during reception can be provided. Although image recognition is used by the position detection units 150 of the acoustic transducers 103 in the present embodiment, this configuration is not restrictive. Any methods which can detect the positions of the acoustic transducers 103 on the flexible film 102 facing the support member 101 may be employed.

### Eighth Embodiment

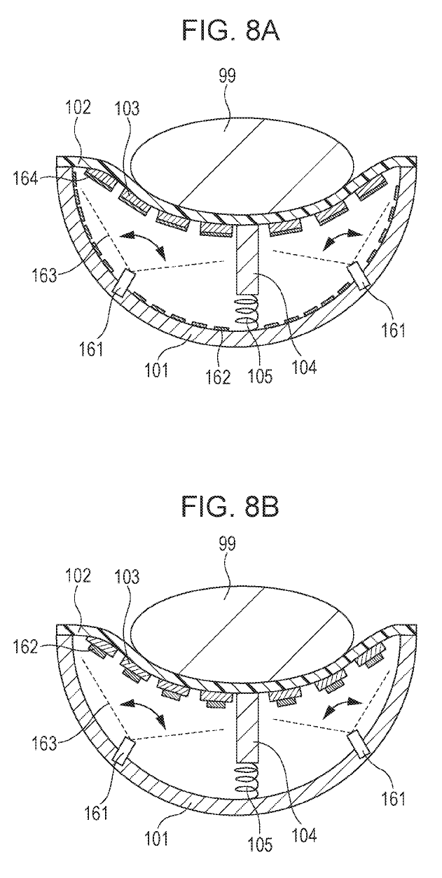

An eighth embodiment relates to a probe of another form having a position detecting function of acoustic transducers 103. Other configurations are the same as those of the seventh embodiment. The eighth embodiment will be described with reference to FIGS. 8A and 8B. In FIG. 8A, the reference numeral 161 denotes a laser scanner, 162 denotes a light-receiving element, 163 denotes laser scanning light, and 164 denotes a mirror. In a form of FIG. 8A of the eighth embodiment, a back side (i.e., a surface on the side opposite to the side on which a subject 99 exists) of each acoustic transducer 103 is irradiated with the laser scanning light 163 while scanning the laser scanning light 163 from a support member 101 side, and position coordinates and an angle of the acoustic transducer 103 are detected based on reflected light.

The form of FIG. 8A includes the laser scanners 161 in the recess of the support member 101 which can scan with varying angles while emitting laser scanning light 163. Plural light-receiving elements 162 are disposed in an array on the entire surface of the recess of the support member 101 in areas where the laser scanners 161 are not disposed. The light-receiving element 162 has a function to simultaneously detect entrance of light into the light-receiving element and an angle of the entered light. Specifically, the light-receiving element 162 may be constituted by a separate type light-receiving element, etc. A back side (i.e., a surface on the side opposite to the side on which a subject 99 exists) of each acoustic transducer 103 is provided with the mirror 164.

The laser scanners 161 emit the laser scanning light 163 toward the entire flexible film 102 to perform scanning. When the laser scanning light 163 reaches a certain acoustic transducer 103, the light is reflected by the mirror 164 disposed on the back side of the acoustic transducer 103, and returns to the recess of the support member 101. By detecting the reflected light with the light-receiving elements 162 arranged in an array, coordinates and an angle of the acoustic transducer 103 can be detected.

In the present embodiment, since the laser scanning light and the light-receiving elements are used, it is not necessary to perform complicated image processing compared with the seventh embodiment. Therefore, the coordinate position and the angle of the acoustic transducer can be detected in a simpler detection process. Therefore, according to the present embodiment, a photoacoustic probe capable of efficiently receiving acoustic waves conforming to various target parts of subjects, lowering load of processing coordinates of acoustic transducers during reception, and grasping coordinates correctly can be provided.

Another form of the present embodiment is described with reference to FIG. 8B. The form of FIG. 8B differs from the form of FIG. 8A in that each acoustic transducer 103 has a light-receiving element 162 on the back side. In the configuration of FIG. 8B, since each acoustic transducer 103 includes the light-receiving element 162, the laser scanning light 163 enters directly the light-receiving element 162, and calculation of a reflection path is unnecessary. Therefore, the detection process of the coordinate position and the angle can be further simplified. According to the another form of the present embodiment, a photoacoustic probe capable of efficiently receiving acoustic waves conforming to various target parts of subjects, further lowering load of processing coordinates of acoustic transducers during reception, and grasping coordinates correctly can be provided.

### Ninth Embodiment

A ninth embodiment relates to a probe provided with another position detection unit of acoustic transducers 103. Other configurations are the same as those of the seventh or the eighth embodiment.

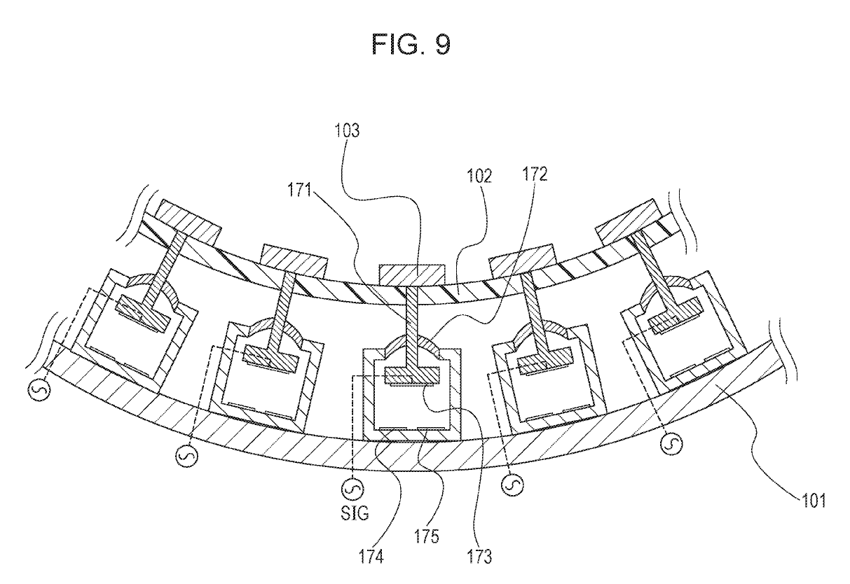

The ninth embodiment will be described with reference to FIG. 9. In FIG. 9, the reference numeral 171 denotes a lever stick, 172 denotes a membrane, 173 denotes a first position detection electrode, 174 denotes a second position detection electrode, 175 denotes a third position detection electrode, and SIG denotes an AC signal source. The ninth embodiment includes the lever stick 171 connected integrally with a back side of each acoustic transducer 103. The coordinates and inclination of the acoustic transducer 103 are detected by detecting the positional relationship between the support member 101 and the lever stick 171.

In FIG. 9, a first position detection electrode 173 is provided on the other side of the lever stick 171. An AC signal is superimposed on the first position detection electrode 173 by the AC signal source SIG. A second position detection electrode 174 and a third position detection electrode 175 are disposed in the recess of the support member 101 to face the first position detection electrode 173. A distance to the first position detection electrode 173 and inclination thereof can be detected by measuring magnitudes of AC signals respectively detected in the second position detection electrode 174 and the third position detection electrode 175 and a ratio between the AC signals. Two electrodes are provided on the side of the support member 101 in FIG. 9. If four electrodes are disposed, angles in the direction of 360 degrees can be detected. In FIG. 9, the lever stick 171 is held by the stretchable and deformable membrane 172, but the membrane 172 is unnecessary if the flexible film 102 can hold plural lever sticks 171.

According to the present embodiment, since the positional and angular relationships between each acoustic transducer 103 and the support member 101 can be detected directly, the position and the angle can be detected more precisely. Therefore, a photoacoustic probe capable of efficiently receiving acoustic waves conforming to various target parts of subjects and grasping coordinates etc. of acoustic transducers during reception more correctly can be provided.

### Tenth Embodiment

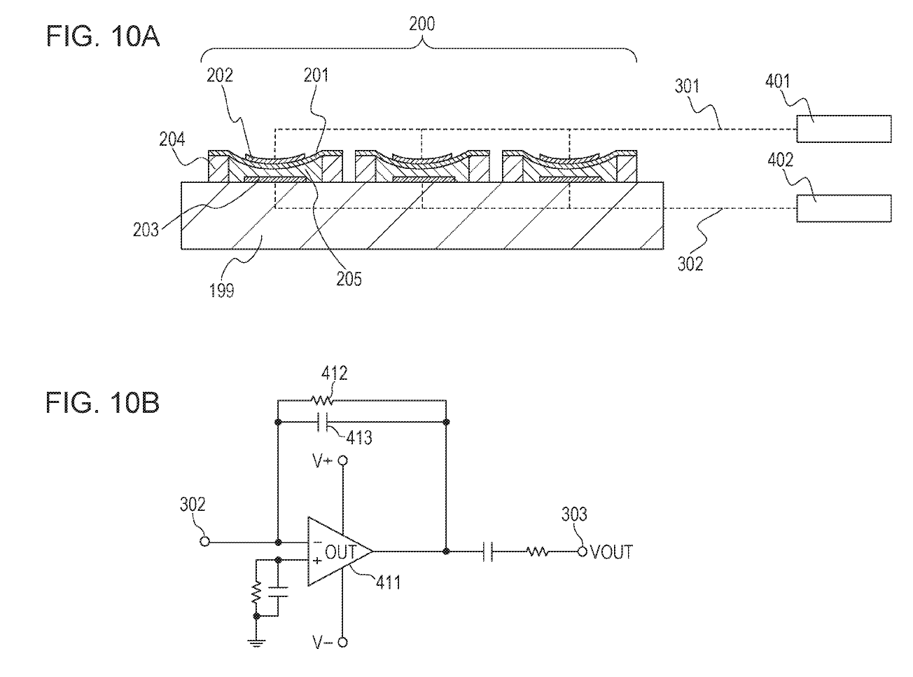

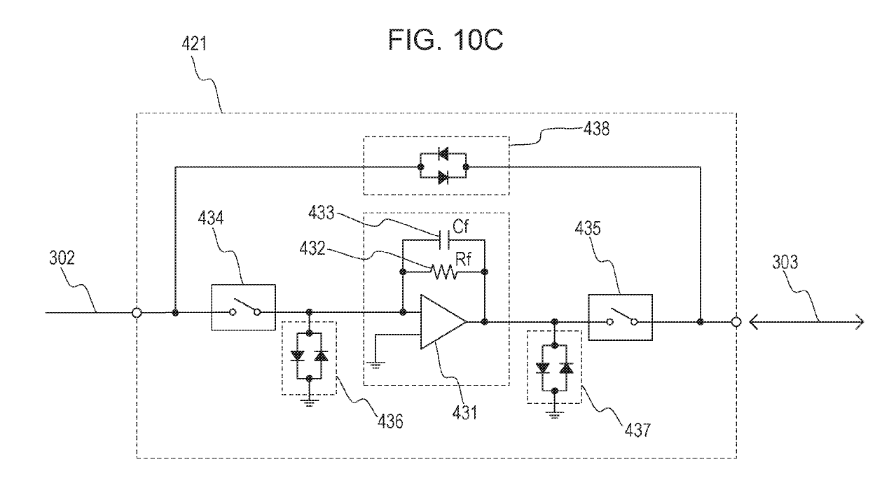

A tenth embodiment relates to a configuration characteristic in a form of an acoustic transducer 103. Other configurations are the same as those of any of the first to the ninth embodiments. FIG. 10A is a schematic diagram and FIGS. 10B and 10C are circuit diagrams illustrating an acoustic transducer 103 related to the tenth embodiment. In FIG. 10A, the reference numeral 199 denotes a chip (a substrate), 201 denotes a vibration film, 202 denotes a first electrode, 203 denotes a second electrode, 204 denotes a support portion, and 205 denotes a gap (cavity). The reference numeral 301 denotes a first wire, 302 denotes a second wire, 303 denotes a third wire, 401 denotes a DC voltage generation unit, and 402 denotes a receiving circuit.

A feature of the present embodiment is that the acoustic transducer 103 is a capacitive transducer 200. The capacitive transducer 200 is produced on the chip 199 of silicon using Micro Electro Mechanical Systems (MEMS) technology adapting a semiconductor process. A capacitive acoustic transducer has significantly greater reception frequency characteristics compared with a piezoelectric acoustic transducer. The vibration film 201 is supported on the chip 199 by the support portion 204, and vibrates in response to a photoacoustic (an ultrasonic) wave. The first electrode 202 is disposed on the vibration film 201, and the second electrode 203 is disposed on the chip 199 at a position to face the first electrode 202. A pair of the first electrode 202 and the second electrode 203 disposed to face other via the vibration film 201 and the gap 205 is referred to as a cell.

In the present embodiment, the first electrode 202 is drawn out of the chip 199 via the first wire 301 and is connected with the DC voltage generation unit 401. The DC voltage generation unit 401 causes several tens to several hundreds of volts of potential difference between the first electrode 202 and the second electrode 203. The second electrode 203 is drawn out of the chip 199 via the second wire 302 and is connected with a receiving circuit 402. When the vibration film 201 and the first electrode 202 vibrate, a distance between the first electrode 201 and the second electrode 203 changes and electrostatic capacity between these electrodes changes. The potential difference between these electrodes generates a minute current corresponding to capacity variation. The minute current is converted into a voltage in the receiving circuit 402 connected with the second electrode 203, and output from the third wire 303 (see FIG. 10B).

Plural cells are disposed on the chip 199, the first electrodes 202 on the chip 199 are electrically connected mutually, and the second electrodes 203 on the chip 199 are also electrically connected mutually. The second electrodes on the chip 199 are connected with different receiving circuit 402 for each chip 199. In an ultrasonic probe of the present embodiment, the same number of receiving circuits 402 and chips 199 are provided, and each chip 199 on which plural capacitive transducers 200 are disposed functions as an independent receiving element (an unit of the receiving element is called an element). The size of the receiving element is in the range of several hundreds of micrometers to several millimeters, and the number of receiving elements is in the range of one hundred to several thousands. In the present embodiment, plural chips 199 are provided on the film 102 which is a support film.

In the present embodiment, since the capacitive transducer 200 is used as the acoustic transducer 103, a range of receiving frequency of a photoacoustic wave is large, and a signal including more information can be acquired from a subject. Therefore, a photoacoustic probe capable of efficiently receiving acoustic waves conforming to various target parts of subjects, and favorably reproducing subject information can be implemented.

A detection circuit of the receiving circuit 402 will be described with reference to FIG. 10B as a more specific form of the present embodiment. A feature of this form is that a transimpedance circuit configuration including an operational amplifier 411 is used in the detection circuit. A resistance 412 and a capacitor 413 are disposed in parallel in a negative feedback portion of the operational amplifier 411, and a current input in a feedback portion is converted into a voltage. With feedback characteristics of the operational amplifier 411, an influence on current-potential conversion efficiency of the parasitic capacitance at input wiring can be reduced by using a broadband operational amplifier. Therefore, excellent receiving characteristics of the ultrasonic wave with reduced deterioration in current-voltage conversion can be obtained compared with a case where the receiving circuit 402 is disposed very close to the capacitive transducer 200 (i.e., a case where the parasitic capacitance of the wiring is very small).

According to the present embodiment, since the transimpedance circuit configuration employing the operational amplifier 411 is used in the detection circuit 402, an influence of the capacitance parasitic on the input terminal of the detection circuit 402 on receiving characteristics is reduced. Therefore, a position of the second wire 302 connected with the detection circuit 402 changes when the flexible film 102 deforms, but, in the present embodiment, receiving characteristics is less affected by an influence of a change in the magnitude of the parasitic capacitance of the second wire 302 caused by the change in the position of the second wire 302. Therefore, a photoacoustic probe with little deterioration of receiving characteristic can be provided.

A feature of another form illustrated in FIG. 10C is that a drive detection circuit 421 is provided instead of the detection circuit. The drive detection circuit 421 has a function not only to detect a photoacoustic wave (an ultrasonic wave) received by the capacitive transducer (CMUT) 200 as a signal, but to illuminate a subject with the ultrasonic wave (i.e., transmit the ultrasonic wave) from the capacitive transducer 200.

In FIG. 10C, the reference numeral 421 denotes a drive detection circuit, 431 denotes an operational amplifier, 432 denotes a feedback resister, 433 denotes a feedback capacitance, 434 and 435 denote high breakdown voltage switches, 436 and 437 denote diodes, and 438 denotes a high breakdown voltage diode. One or more elements of the capacitive transducer 200 are disposed on a single chip, and the second electrode 203 of the capacitive transducer 200 is connected with the drive detection circuit 421. The drive detection circuit 421 has a function to apply, to the capacitive transducer 200, high voltage pulses used for transmission of the ultrasonic wave from an apparatus, and output a minute current from the capacitive transducer 200 as detection signals to the apparatus.

FIG. 10C is a circuit diagram illustrating the drive detection circuit 421. The feedback resister 432 and the feedback capacitance 433 are disposed in parallel at the negative feedback portion of the operational amplifier 431, and have a function to perform current-voltage conversion. The high breakdown voltage switches 434 and 435 and diodes 436 and 437 are connected with an input terminal and an output terminal of the operational amplifier 431, respectively. The high breakdown voltage diode 438 disconnects wire connection between terminals when the voltage between the terminals becomes a predetermined value or smaller (i.e., 1V or smaller). When applied a voltage higher than a predetermined voltage (i.e., about several volts), the high breakdown voltage switches 434 and 435 disconnect wire connection between I/O terminals of the switches. When no high voltage pulse for transmission is applied, there is substantially no potential difference between the terminals, and therefore the wire connection at the I/O terminals in the high breakdown voltage diode 438 is disconnected. Wires between the switches in the high breakdown voltage switches 434 and 435 are connected since no high voltage is applied from the outside. Therefore, the minute current from a transducer can be converted into a voltage in the operational amplifier 431, and detection signals can be output to an apparatus connected with the outside (not illustrated).

When high voltage pulses for transmission are applied from an apparatus (not illustrated), the wires inside the high breakdown voltage diode 438 are connected, and a voltage higher than a predetermined voltage (i.e., about several volts) is applied to the high breakdown voltage switches 434 and 435. The high breakdown voltage switches 434 and 435 disconnect the wire connection thereinside. Therefore, damage to the operational amplifier 431 due to application of a high voltage thereto can be prevented. Since a signal output from the operational amplifier 431 is cut by the high breakdown voltage switch 435, no influence is exerted on the high voltage pulses applied for the transmission.

Therefore, the high voltage pulses for transmitting the ultrasonic wave can be applied to the second electrode 203 of the transducer.

According to this form, since a capacitive transducer with a wide range frequency characteristics can transmit, in addition to receive, ultrasonic waves, subject information by transmission and reception of ultrasonic waves with respect to a subject in addition to reception of photoacoustic waves can be acquired. Therefore, an acoustic probe capable of acquiring more detailed subject information can be provided.

### Eleventh Embodiment

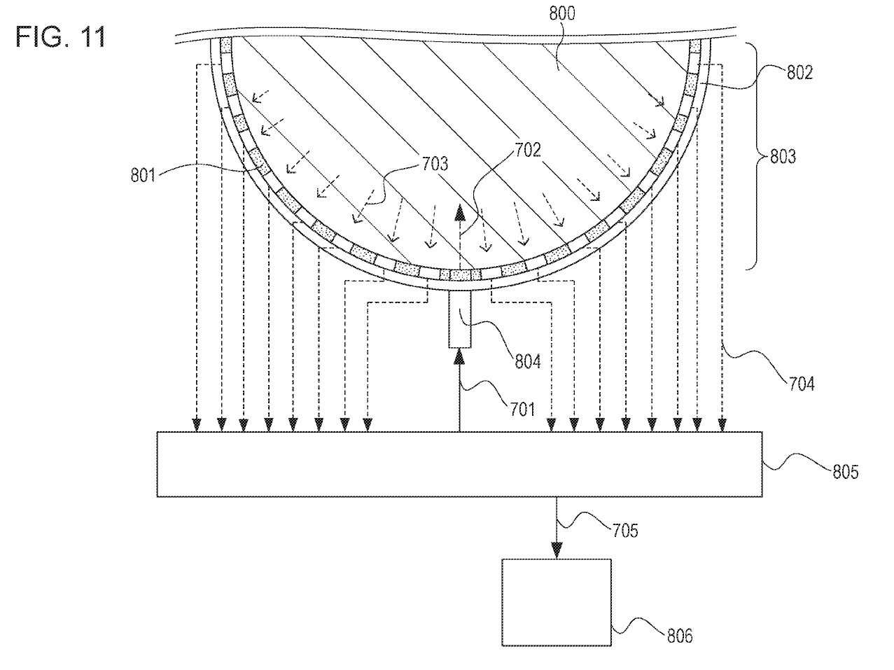

The probe according to any of the first to the tenth embodiments can be used for the reception of a photoacoustic wave (an ultrasonic wave) using a photoacoustic effect, and is applicable to a subject information acquisition apparatus illustrated in FIG. 11. The probe is illustrated in a simplified manner in FIG. 11. In FIG. 11, a flexible film 802, an acoustic transducer 803, and a light source 804 are illustrated, whereas a support member is not.

An operation of the subject information acquisition apparatus of the present embodiment will be described with reference to FIG. 11. First, in accordance with a light emission instruction signal 701, a light source 804 (104) generates light (pulsed light) 702 for illuminating a measurement target (a subject) 800 (99). When the light 702 is illuminated, a photoacoustic wave (an ultrasonic wave) 703 is generated in the measurement target 800 and the ultrasonic wave 703 is received by the plural acoustic transducers 803 disposed on the flexible film 802 (102) of the acoustic probe. A space between the flexible film 802 and the subject 800 is filled with an ultrasonic gel 801 in order to prevent attenuation of the acoustic wave (the ultrasonic wave) caused by air bubbles.

Information about the magnitude, shape, and time of a reception signal is transmitted as photoacoustic wave reception signals 704 to an image information generation apparatus 805 which is a signal processing unit.

Information about the magnitude, shape, and time of the light 702 emitted from the light source 804 (light emission information) is stored in the image information generation apparatus 805 of photoacoustic signals. In the image information generation apparatus 805 of the photoacoustic signals, image signals of the measurement target 800 are generated based on the photoacoustic wave reception signals 703 and the light emission information, and output as reproduction image information 705 by the photoacoustic signals. An image display unit 806 displays the measurement target 800 as an image based on the reproduction image information 705 by the photoacoustic signals. When the acoustic probe of the seventh embodiment is used, since position information of the acoustic transducer can be acquired, more correct subject information can be acquired based on information with the position information.

The photoacoustic (ultrasonic) probe according to the present embodiment can efficiently receive acoustic waves conforming to various target parts of subjects, and therefore can generate high quality images.

### Twelfth Embodiment

The acoustic (ultrasonic) probe described in any of the first to the tenth embodiments can receive a photoacoustic wave (an ultrasonic wave) using a photoacoustic effect and, in addition, transmit a ultrasonic wave to a subject and receive a reflected ultrasonic wave. This probe is applicable to a subject information acquisition apparatus which acquires information about a subject based on the acquired signal. Here, the acoustic probe of the present disclosure receives photoacoustic waves generated by photoacoustic effect in the subject, and transmits and receives ultrasonic waves to and from the subject so as to acquire subject information.

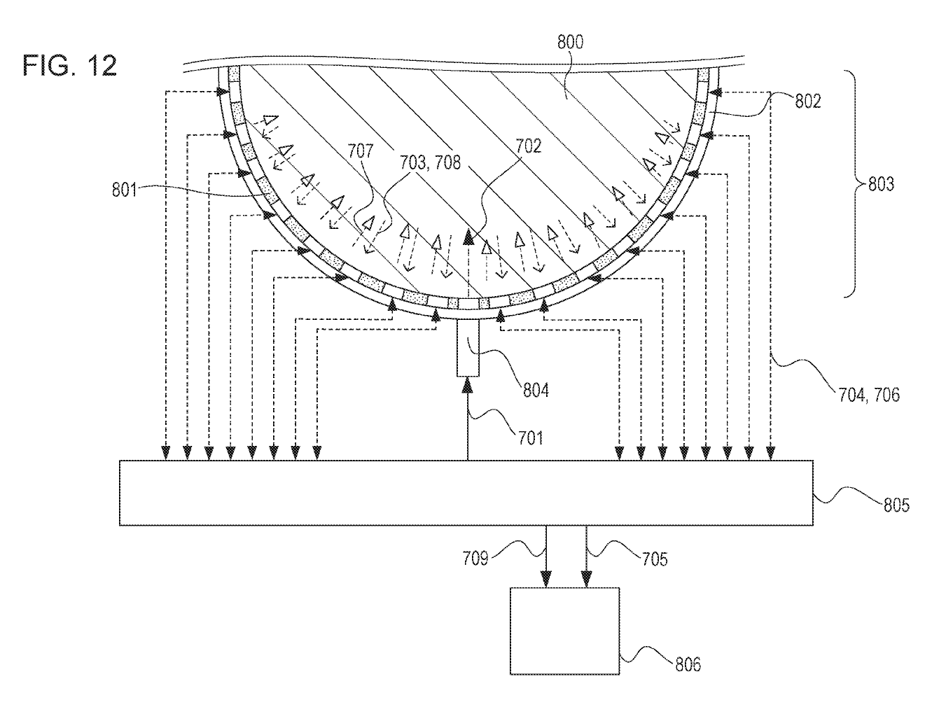

FIG. 12 schematically illustrates the subject information acquisition apparatus according to the present embodiment. In FIG. 12, the reference numeral 706 denotes transmission and reception signals of an ultrasonic wave, 707 denotes a transmitted ultrasonic wave, 708 denotes a reflected ultrasonic wave, and 709 denotes reproduction image information by transmission and reception of a ultrasonic wave. A portion corresponding to the support member 101 is not illustrated in FIG. 12. A probe described in any on the first to the tenth embodiments is used.

The subject information acquisition apparatus of the present embodiment receives the photoacoustic wave and, in addition, performs pulse echo (i.e., transmission and reception of ultrasonic waves) to form an image. Since reception of the photoacoustic wave is the same as that of the eleventh embodiment, the pulse echo (i.e., transmission and reception of ultrasonic waves) will be described below. Based on an ultrasonic transmission signal 706, the ultrasonic wave 707 is output (transmitted) toward the measurement target 800 from plural acoustic transducers 803 disposed on a flexible film 802 of the acoustic probe. The ultrasonic wave is reflected inside the measurement target 800 due to a difference of a specific acoustic impedance of objects exist therein. The reflected ultrasonic wave 708 is received by the plural acoustic (ultrasonic) transducers 803, and information about the magnitude, shape, and time of a reception signal are transmitted to an image information generation apparatus 805 as an ultrasonic reception signal 706. A space between the flexible film 802 and the subject 800 is filled with an ultrasonic gel 801 in order to prevent attenuation of the acoustic wave (ultrasonic wave). The information about the magnitude, shape, and time of the transmitted ultrasonic wave is stored in the image information generation apparatus 805 as ultrasonic transmission information. The image information generation apparatus 805 generates image signals of the measurement target 800 in accordance with the ultrasonic reception signal 706 and ultrasonic transmission information, and outputs the image signals as reproduction image information 709 by transmission and reception of the ultrasonic wave.

The image display unit 806 displays the measurement target 800 as an image based on two pieces of information, i.e., the reproduction image information 705 by the photoacoustic signal and the reproduction image information 709 by transmission and reception of the ultrasonic wave. Since the photoacoustic (ultrasonic) probe according to the present embodiment does not easily deteriorate in the characteristics of the acoustic transducer due to attachment to a subject, a photoacoustic wave can be acquired correctly, and an ultrasonic wave can be correctly transmitted and received with the same probe. Therefore, a high quality photoacoustic image and an ultrasonic image having the same coordinate system can be generated.

In the above embodiment, the transducer may at least receive an ultrasonic wave from a subject, and a signal processing unit may acquire information about the subject using an ultrasonic reception signal from the transducer. Although a capacitive transducer may transmit an ultrasonic wave toward a subject, another transducer may transmit an ultrasonic wave. Only an ultrasonic wave may be received without receiving a photoacoustic wave. As described above, an acoustic probe can detect a photoacoustic wave and/or an ultrasonic wave from a subject located at a position corresponding to a recessed (e.g., hemispherical) support member, and a signal processing unit can generate a living body tissue image of a subject, etc. from a photoacoustic and/or ultrasonic signal acquired by the acoustic probe.

### Thirteenth Embodiment

The acoustic probe described in each of the seventh to the ninth embodiments, which are provided with the position detection units 150, can generate an image based on detected information if the acoustic probe is applied to a subject information acquisition apparatus.

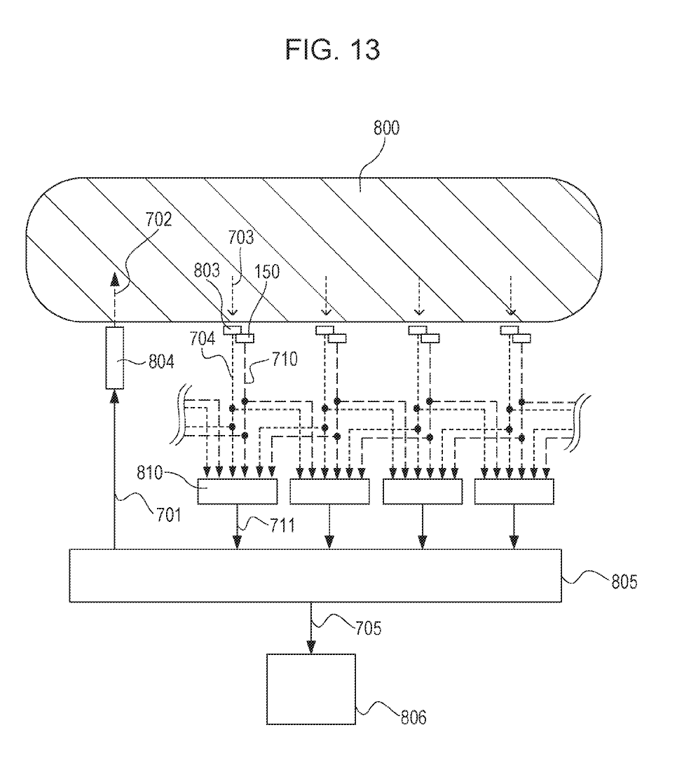

The present embodiment will be described with reference to FIG. 13. In FIG. 13, the reference numeral 150 denotes a position detection unit, 810 denotes a position correction unit of a reception signal, 710 denotes a position detection signal, and 711 denotes a position corrected reception signal.

The acoustic probe of the present disclosure includes the detection units 150 each grasping a position of each acoustic transducer 803. The acoustic probe can always grasp the positions of the acoustic transducers 803 even if a surface shape of a subject changes and positions of the acoustic transducers 803 change.

The position detection unit 150 outputs position information about each acoustic transducer 803 as the position detection signal 710. A reception signal 704 from each acoustic transducer 803 is input in each position correction unit 810 of a reception signal.

In addition to the position detection signal 710 from a target acoustic transducer 803, a position detection signal 710 from an adjoining transducer 803 are input in the position correction unit 810 of a reception signal as position information about the acoustic transducer 803. Further, in addition to the reception signal 704 from a target acoustic transducer 803, a reception signal 704 from an adjoining transducer 803 is input as a reception signal 704 from the acoustic transducer 803. Based on the position detection signal 710 and the reception signal 704 of the target acoustic transducer 803 and, in addition, a position detection signal 710 and a reception signal 704 of an adjoining acoustic transducer 803, a position corrected reception signal 711 is generated in the position correction unit 810 of a reception signal and output. In particular, points for calculation are set on a certain predetermined curved surface, a signal to be received at each point is estimated based on information about positional displacement from these positions and information about a reception signal from each acoustic transducer 803, and the estimated signal is output as the position corrected reception signal 711. In a rough sense, signal delay time is calculated from position information, and the magnitude, frequency characteristics, etc. of the signal are calculated from the reception signal.

Then, in the image information generation apparatus 805, image information is generated based on the position corrected reception signal 711. Since the position corrected reception signal 711 is already converted into a reception signal at a point on a predetermined curved surface, even if a surface shape of a subject changes, it is not necessity to change a process of generating an image.

According to the subject information acquisition apparatus of the present embodiment, since the transducer has a position detection unit, a position and an angle of each transducer can be grasped even if a surface shape of a subject changes and a position of the transducer changes when the subject moves. An image is generated based on the detected information. Therefore, a subject information acquisition apparatus which is less easily affected by a change of a surface shape of a subject and which acquires high quality subject information can be provided.

Although the position information of the transducer is reflected based on the detected position information by converting into a reception signal at a predetermined point on a curved surface in this specification, the present disclosure is not limited to this configuration. Any other methods for correcting a position of a transducer based on detected position information and generating an image may be used similarly.

### Advantageous Effects of the Present Disclosure

The acoustic probe of the present disclosure can move acoustic transducers conforming to various target parts of subjects, and can efficiently receive acoustic waves.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2015-244076, filed Dec. 15, 2015, which is hereby incorporated by reference herein in its entirety.

What is claimed is:

1. An acoustic probe comprising:
at least two acoustic transducers;
a support film in which the at least two acoustic transducers are disposed;
a recessed support member configured to support the support film; and
a position detection unit configured to acquire positions information of each of the at least two acoustic transducers with respect to the support member,
wherein the support film is deformable so that the at least two acoustic transducers are moved conforming to a shape of a subject.

2. The acoustic probe according to claim 1, further comprising an illumination unit configured to illuminate the subject with light, wherein the illumination unit is supported by the support film.

3. The acoustic probe according to claim 1, wherein the support member is semi-cylindrical or semi-spherical in shape.

4. The acoustic probe according to claim 1, further comprising a holder configured to hold the subject when the subject is pressed against the support film.

5. The acoustic probe according to claim 4, wherein the holder is a pedestal fixed to the support member.

6. The acoustic probe according to claim 4, wherein the support film is disposed to be nipped between the holder and the subject.

7. The acoustic probe according to claim 6, wherein the holder is disposed in a space between the support film and the support member.

8. The acoustic probe according to claim 7, wherein the holder is a bag filled with gas.

9. The acoustic probe according to claim 1, wherein two pairs of support films in which the at least two acoustic transducers are disposed and recessed support members configured to support the support films are provided, and the two pairs are mutually movable so that the subject can be nipped by the support films.

10. The acoustic probe according to claim 4, further comprising a lid provided to be movable with respect to the support member, wherein the holder is disposed inside the lid and in a space between the support film and the support member.

11. The acoustic probe according to claim 10, wherein the holder is a bag filled with gas.

12. The acoustic probe according to claim 1, wherein a space is formed between the support film and the recessed support member.

13. The acoustic probe according to claim 1, wherein the position detection unit includes a camera for acquiring an image.

14. The acoustic probe according to claim 13, wherein each of the at least two acoustic transducers has an image-identification pattern for identifying its own self.

15. The acoustic probe according to claim 1, wherein the position detection unit includes a laser scanner and light-receiving elements attached to the at least two acoustic transducers.

16. The acoustic probe according to claim 1, wherein the position detection unit includes a laser scanner, mirrors attached to the at least two acoustic transducers, and light-receiving elements attached to the support member.

17. The acoustic probe according to claim 1, wherein the position detection unit includes position detection electrodes attached to the at least two acoustic transducers, and position detection electrodes attached to the support member.

18. The acoustic probe according to claim 1, wherein the at least two acoustic transducers are capacitive transducers.

19. The acoustic probe according to claim 18, wherein a detection circuit which includes a transimpedance circuit using an operational amplifier and which detects a current when the at least two acoustic transducers receive an acoustic wave is connected with the acoustic transducers.

20. The acoustic probe according to claim 18, wherein a drive detection circuit which includes a circuit for detecting a current when the at least two acoustic transducers receive an acoustic wave and which transmits and receives a signal related to an acoustic wave is connected with the at least two acoustic transducers.

21. A subject information acquisition apparatus comprising:
the acoustic probe according to claim 1; and
a signal processing unit configured to convert a signal detected by the acoustic probe into a signal representing information about a subject.

22. The subject information acquisition apparatus according to claim 21, wherein the signal processing unit converts a signal detected by the acoustic probe into an image signal of a subject.

23. The subject information acquisition apparatus according to claim 21, wherein the acoustic probe detects a photoacoustic wave from a subject generated by a photoacoustic effect.

24. The subject information acquisition apparatus according to claim 21, wherein the acoustic probe detects an ultrasonic wave from a subject.

25. The subject information acquisition apparatus according to claim 24, wherein the acoustic probe transmits and receives an ultrasonic wave.

26. The subject information acquisition apparatus according to claim 21, wherein the signal processing unit acquires a signal detected by the acoustic probe and position information about the at least two acoustic transducers, and acquires information about a subject based on these information.
